SEQUENCE LISTING (21 sequences)

SEQ ID NO: 1 - DNA, 40 bp, Artificial Sequence
  Synthetic Sequence
gggaatttccgggaatttccgggaatttccgggaatttcc

SEQ ID NO: 2 - DNA, 60 bp, Artificial Sequence
  Synthetic Sequence
gagtatgtctagactgagtatgtctagactgagtatgtctagactgagtatgtctagact

SEQ ID NO: 3 - DNA, 78 bp, Artificial Sequence
  Synthetic Sequence
tgcattcccgtaatgcattcccgtaatgcattcccgtaatgcattcccgtaatgcattcccgtaatgcattcccgtaa

SEQ ID NO: 4 - DNA, 20 bp, Artificial Sequence
  Synthetic Sequence
ggctggagcatccacgtgtt

SEQ ID NO: 5 - DNA, 19 bp, Artificial Sequence
  Synthetic Sequence
tggtgacaaaaactacagc

SEQ ID NO: 6 - DNA, 20 bp, Artificial Sequence
  Synthetic Sequence
aggtcaaaggtttggaagca

SEQ ID NO: 7 - DNA, 20 bp, Artificial Sequence
  Synthetic Sequence
tgaagcagctatggcaactg

SEQ ID NO: 8 - DNA, 23 bp, Artificial Sequence
  Synthetic Sequence
cggtgcctatgtctcagcctctt

SEQ ID NO: 9 - DNA, 23 bp, Artificial Sequence
  Synthetic Sequence
aggccatttgggaacttctcatc

SEQ ID NO: 10 - DNA, 26 bp, Artificial Sequence
  Synthetic Sequence
agaaaagagttgtgcaatggcaattc

SEQ ID NO: 11 - DNA, 24 bp, Artificial Sequence
  Synthetic Sequence
aataggcaaatttcctgattatat

SEQ ID NO: 12 - DNA, 20 bp, Artificial Sequence
  Synthetic Sequence
gcctatcgccaatgagctgc

SEQ ID NO: 13 - DNA, 21 bp, Artificial Sequence
  Synthetic Sequence
cttgagtgtggctatgacttc

SEQ ID NO: 14 - DNA, 21 bp, Artificial Sequence
  Synthetic Sequence
ggagttcgaggaaccctagtg

SEQ ID NO: 15 - DNA, 21 bp, Artificial Sequence
  Synthetic Sequence
gggatttgtagtggatcgtgc

SEQ ID NO: 16 - DNA, 20 bp, Artificial Sequence
  Synthetic Sequence
caaaagtaactgccgaagca

SEQ ID NO: 17 - DNA, 20 bp, Artificial Sequence
  Synthetic Sequence
ctgagctagggaggacaagg

SEQ ID NO: 18 - DNA, 18 bp, Artificial Sequence
  Synthetic Sequence
cagaagcagaatgtgacc

SEQ ID NO: 19 - DNA, 22 bp, Artificial Sequence
  Synthetic Sequence
gtagtagtagaatggagatagg

SEQ ID NO: 20 - DNA, 22 bp, Artificial Sequence
  Synthetic Sequence
gggctgccatttgcagtggcaa

SEQ ID NO: 21 - DNA, 22 bp, Artificial Sequence
  Synthetic Sequence
tgttagtggggtctcgctcctg

(19) United States
(12) Patent Application Publication
(10) Pub. No.: US 2024/0226214 A1
(43) Pub. Date: Jul. 11, 2024

(54) METHODS FOR TREATING INFLAMMATORY AND FIBROTIC DISEASES AND DISORDERS

(71) Applicant: YALE UNIVERSITY, New Haven, CT (US)

(72) Inventors: YungChi Cheng, Woodbridge, CT (US); Wing Lam, New Haven, CT (US)

(21) Appl. No.: 17/995,072

(22) PCT Filed: Mar. 29, 2021
(86) PCT No.: PCT/IB2021/000963

§ 371 Date: Sep. 29, 2022

Related U.S. Application Data
(60) Provisional application No. 63/001,693, filed on Mar. 30, 2020.

(51) Int. Cl. A61K 36/185; A61K 36/07; A61K 36/24; A61K 36/28; A61K 36/282; A61K 36/41; A61K 36/48; A61K 36/484; A61K 36/537; A61K 36/539; A61K 36/65; A61K 36/739; A61K 36/74; A61K 36/82; A61K 45/06; A61P 29/00

(52) U.S. Cl. CPC A61K 36/185; A61K 36/07; A61K 36/24; A61K 36/28; A61K 36/282; A61K 36/41; A61K 36/48; A61K 36/484; A61K 36/537; A61K 36/539; A61K 36/65; A61K 36/739; A61K 36/74; A61K 36/82; A61K 45/06; A61P 29/00

(57) ABSTRACT

The present invention relates to methods of using herbal extracts for treating and preventing inflammatory and fibrotic diseases and disorders. Additionally, the herbal extracts of the present invention are also useful for treating diseases and disorders that are caused by bacterial, viral, or fungal infections.

### CROSS-REFERENCE TO RELATED APPLICATION

This application is a 35 U.S.C. § 371 national phase application of, and claims priority to, International Application No. PCT/IB2021/000963, filed Mar. 29, 2021, which claims the benefit of priority to U.S. Provisional Patent Application Ser. No. 63/001,693 entitled “METHODS FOR TREATING INFLAMMATORY AND FIBROTIC DISEASES AND DISORDERS,” filed Mar. 30, 2020, the disclosure of which is incorporated herein by reference in their entireties.

### REFERENCE TO SEQUENCE LISTING

The Sequence Listing concurrently submitted herewith as a xml file named “047162-7283US1_ST26.xml,” created on Sep. 23, 2022 and having a size of 27,448 bytes is herein incorporated by reference pursuant to 37 C.F.R. § 1.52(e)(5).

### BACKGROUND OF THE INVENTION

Lung infections caused by virus, bacteria, and fungi can lead to lung inflammation and fibrosis. For example, viral infections, COVID-19, MERS-COV, SARS-coV, can cause severe lung inflammation that can lead to pneumonia and fibrosis. Bacterial infections, which are very common in elderly population, kids, and cancer patients, can also cause pneumonia and sepsis. Other factors that can cause lung inflammation and/or lung cancer include air pollution and smoking. Liver and kidney inflammation and fibrosis can also be caused by viral and other microbial infections.

Uncontrolled lung inflammation can generate “cytokine storm” which leads to organ failure and death. It is known that TNF-α, IL-1β, IL6, TLRs are the key mediators for generation of cytokine storm. Current treatments for fighting “cytokine storm” include specific antibodies (such as anti-TNF-α) and high dose steroid treatment but the results are not very satisfying. Further, it is well known that TGF-β signaling plays a key role in lung, kidney and liver fibrosis. Anti-IL6 antibody has pronounced activity against COVID-19 associated lung pneumonia. Also, it is known that anti-IL-1β antibody can significantly reduce incident lung cancer and lung cancer mortality. However, the current single chemical and single target approach cannot simultaneously inhibit multiple targets and has only limited effects on inflammatory and fibrotic diseases. In order to treat multiple diseases and disorders simultaneously, there is need in the art to develop agents that have potential to inhibit activities of multiple targets including TGF-β, TNF-α, and/or IL-1β, and/or IL6. The present disclosure addresses this unmet need.

### BRIEF SUMMARY OF THE INVENTION

In one aspect, a method of treating or preventing a disease or disorder in a subject is provided. The method includes administering to the subject in need thereof a therapeutically effective amount of composition comprising at least one herbal extract or any active chemical present in the at least one herbal extract, wherein the at least one herbal extract is from an herb selected from the group consisting of: Caesalpinia sappatin L (A16), Salvia miltiorrhiza Bge (B8), Glycyrrhiza uralensis Fisch (G), Punica granatum L (PM), Rubia cordifolia L. (Y1830), Inula japonica, (capitulum) (P8), Antrodia camphorata (Y1646), Apocynum venetum L (F1), Eurycoma longifolia (TK), Paeonia veitchii (X7), Sanguisorba officinalis L (E5), Artemisia argyi Levl. et Vant. (U1), Camellia sinensis var. assamica (PE), Rhodiola rosea L (HJT-1933), Taxillus sutchuenensis (Lecomte) Danser (Z-12), total glucosides of white peony (T-P), and Scutellaria baicalensis Georgi (E1); and wherein the disease or disorder is (a) related to the activity of TGF-β (and SMAD2/3) and/or IL-1B and/or TNF-α (and NF-kB), and/or IL6 (and STAT3), (b) is caused by bacterial, fungal or viral infection, or (c) is the result of inflammation and/or fibrosis.

### BRIEF DESCRIPTION OF THE DRAWINGS

The following detailed description of specific embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIG. 1 is a table showing inhibitory activity of the selected herbs on IL-1β/TNF-α and/or TGF-β induced NF-kB (p50/p65) or SMAD2/3 mediated transcriptional activity of A549 cells and IL6 induced STAT3 mediated transcriptional activity. Values are IC50 (dose of herbal extract required for 50% inhibition of the luciferase activity as comparing to ligand alone control group). Stronger inhibition (or smaller IC50) is highlighted as green while weaker inhibition (larger IC50) is highlighted as colorless, as shown at the bottom of the table. No effect or IC50>750 g/mL is marked with “=” in the table.

FIGS. 2A-2B are graphs illustrating enhancement of TNF-α/IL-1β on TGF-β induced SMAD2/3 mediated transcriptional activity of (FIG. 2A) A549 and (FIG. 2B) HEK293 reporter cells. (FIG. 2A) Relative luciferase activity of A549 SMAD2/3-reporter cells following 6 h-treatment of TGF-β (2.5 ng/mL), TNF-α (10 ng/mL), IL-1B (2 ng/mL), LPS (1 μg/mL), PGN (1 μg/mL), ZYM (1 μg/mL), IL6 (20 ng/mL), IFNa (20 ng/mL), IFNa (20 ng/mL), IFNb (20 ng/mL) or TGF-β (2.5 ng/ml) plus other ligands. (FIG. 2B) Relative luciferase activity of HEK293 SMAD2/3-reporter cells following 6 h-treatment of TGF-β (2.5 ng/mL) or TGF-β (2.5 ng/ml) plus TNF-α (10 ng/mL), IL-1B (2 ng/mL).

FIGS. 3A-3C illustrate inhibitory effects of the selected herbs on COX2 and ICAM protein expression induced by IL-1β and TNF-α of A549 cells. Western blot for detecting COX and ICAM of A549 cells following co-treatment of IL-1β (FIG. 3A) or TNF-α (FIG. 3B) with the selected herbs. (FIG. 3C) Summary for the inhibitory effect (=no effect, +weak inhibition, +++strong inhibition) of active herbs on COX and ICAM protein expression of A549 cells induced by IL-1β or TNF-α for 24 h. GAPDH was used as protein loading control for comparison.

FIGS. 4A-4C illustrate inhibitory effects of the selected herbs on LOXL2 and TGM2 protein expression induced by TGF-β of A549 cells. Western blot for detecting LOXL2 (FIG. 4A) and TGN2 (FIG. 4B) of A549 cells following co-treatment of TGF-β (2.5 ng/ml) with the selected herbs for 24 h. (FIG. 4C) Summary for the inhibitory effect (=no effect, +weak inhibition, +++strong inhibition) of active herbs on LOXL2 or TGM2 protein expression of A549 cells induced by TGF-β for 24 h. GAPDH was used as protein loading control for comparison.

FIGS. 5A-5B illustrate inhibitory effects of the selected herbs on COX2, IL-1β protein expression induced by LPS of TPA TPA differentiated TPH1 cells. (FIG. 5A) Western blot for detecting COX2 and IL-1β of TPA differentiated TPH1 cells following co-treatment of LPS with the selected herbs for 24 h. (FIG. 5B) Summary for the inhibitory effect (=no effect, +weak inhibition, +++strong inhibition) of the active herbs on COX2, IL-1 protein expression induced by LPS of TPH1.

FIG. 6 is a table depicting heat map for inhibitory effects of the selected herbs on the pro-inflammatory cytokine/chemokine secretion from THP1 following co-treatment of LPS (1 μg/mL) and selected herbs for 24 h. TPH1 cells were differentiated by TPA (10 nM) for 48 h. After exchanging culture medium without TPA, the differentiated TPH1 cells were co-treated with LPS (1 ug/ml) and the selected herbs for 24 h. Cytokine/Chemokines of culture medium (pg/ml) were determined by bead array analysis. Values are relative level of cytokines/chemokines of LPS+herb groups as comparing to LPS alone group. Number of the last column indicated the number of inhibited (>50%) cytokine/chemokines out of the detectable cytokine/chemokines of the experiment.

FIGS. 7A-7B are a set of table showing inhibitory effects of the selected herbs on pro-inflammatory gene mRNA expression of different tissues induced by LPS. Total RNA from different tissues of BDF1 mice were extract following LPS (2.5 mg/kg, IP, once) or LPS co-treated with the selected herbs (1 g/kg, BID, PO) treatment for 24 h (N=3 to 6). Relative pro-inflammatory cytokines mRNA were determined for MCP1, iNOS, CXCL1, CXCL9, CXCL10, IL-1β, TNF-α, IL6 GAPDH using qRT-PCR. GAPDH was used as internal control. Values showed the relative mRNA expression comparing to LPS only treatment group. Highlight Green showed P<0.05 in T-test analysis.

FIGS. 8A-8H are graphs illustrating inhibitory effects of the selected herbs on pro-inflammatory gene mRNA expression of lung tissues induced by LPS. Total RNA from lung tissues of BDF1 mice were extract following LPS (2.5 mg, IP, once) or plus the selected herbs (1 g/kg, BID, PO) treatment for 24 h (N=3 to 6). Relative pro-inflammatory cytokines mRNA expression was determined for MCP1 (FIG. 8A), IL-1β (FIG. 8B), TNF-α (FIG. 8C), IL6 (FIG. 8D), CXCL1 (FIG. 8E), CXCL9 (FIG. 8F), CXCL10 (FIG. 8G), iNOS (FIG. 8H) using qRT-PCR. GAPDH was used as internal control. Values showed the relative mRNA expression comparing to LPS only treatment group. *=P<0.05 in T-test analysis.

FIGS. 9A-9G are graphs illustrating inhibitory effects of the selected herbs on pro-inflammatory protein expression of plasma induced by LPS. BDF1 mice was treated with LPS (2.5 mg/kg, IP, once), LPS plus the selected herbs (0.5 g/kg, BID, PO) treatment for 24 h (N=3 to 6), plasma was taken for cytokine/chemokine determination using bead array assays. Relative pro-inflammatory cytokines proteins were determined for MCP1 (FIG. 9A), IL6 (FIG. 9B), CXCL9 (FIG. 9C), G-CSF (FIG. 9D), IL-1β (FIG. 9E), TNF-α (FIG. 9F) using bead arrays. *=P<0.05 in T-test analysis.

FIGS. 10A-10G are graphs illustrating inhibition of LPS induced blood count changes by U1 and Y1830. BDF1 mice was treated with LPS (2.5 mg/kg, IP, once), LPS plus U1 or Y1830 (0.5 g/kg, BID, PO) treatment for 24 h (N=3 to 6), blood was taken for complete blood count (FIG. 10A: WBC=white blood cells, FIG. 10B: Thrombocytes, FIG. 10C: Lymphocytes, FIG. 10D: Neutrophils, FIG. 10E: Monocytes, FIG. 10F: Eosinophils, FIG. 10G: Basophils) using hematology analyzer. *=P<0.05 in T-test analysis.

### DETAILED DESCRIPTION OF THE INVENTION

The present invention relates to method of using herbal compositions with systemic and multiple targets for treating inflammatory and fibrotic diseases and disorders in lung, kidney and liver. Additionally, the compositions described in the present invention are also useful for treating diseases and disorders caused by bacterial, viral, or fungal infections. The invention provides an improved approach using a system biology approach in which polychemicals from a herb (or combination of herbs) or active components from a herbs (or combination of herbs) can simultaneously inhibit multiple targets. These agents are useful for treating inflammatory and fibrotic diseases as well as for chemoprevention. Examples of diseases and disorders that can be treated using multiple target approach include lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art. It should be understood that the order of steps or order for performing certain actions is immaterial, so long as the present teachings remain operable. Moreover, two or more steps or actions can be conducted simultaneously or not.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

In one aspect, the terms “co-administered” and “co-administration” as relating to a subject refer to administering to the subject a compound and/or composition of the invention along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition can be formulated in any kind of combinations as mixtures of solids and liquids under a variety of solid, gel, and liquid formulations, and as a solution.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “extract” refers to a concentrated preparation or solution of a compound or drug derived from a naturally occurring source, such as an herb or other plant material. Extracts can be prepared by a number of processes including steeping an herb in solution or drying and grinding an herb into a powder and dissolving the powder in a solution. An extract can be further concentrated by removing a portion of the solvent after dissolving an amount of the desired compound in the solution. An extract may also be strained or centrifuged to remove any solid material from the solution.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or downregulate a protein, a gene, and an mRNA stability, expression, function and activity, e.g., antagonists.

The terms “patient,” “subject” or “individual” are used interchangeably herein, and refer to any animal, or cells thereof whether in vitro or in situ, amenable to the methods described herein. In a non-limiting embodiment, the patient, subject or individual is a human. In other embodiments, the patient is a non-human mammal including, for example, livestock and pets, such as ovine, bovine, porcine, canine, feline and murine mammals. In yet other embodiments, the patient is an avian animal or bird. Preferably, the patient, individual or subject is human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “prevent,” “preventing” or “prevention,” as used herein, means avoiding or delaying the onset of symptoms associated with a disease or condition in a subject that has not developed such symptoms at the time the administering of an agent or compound commences.

A “therapeutic” treatment is a treatment administered to a subject who exhibits signs of pathology, for the purpose of diminishing or eliminating those signs.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

The following abbreviations are used herein NF-κB=nuclear factor kappa-light-chain-enhancer of activated B cells; TNF-α=Tumor necrosis factor alpha; IL-1β=Interleukin 1 beta, IL6=Interleukin 6; TLRs=Toll-like receptors; A16=Caesalpinia sappatin L; B8=Salvia miltiorrhiza Bge; G=Glycyrrhiza uralensis Fisch; PM=Punica granatum L; Y1830=Rubia cordifolia L.; P8=Inula japonica, (capitulum); Y1646=Antrodia camphorata; F1=Apocynum venetum L, TK=Eurycoma longifolia; X7 Paeonia veitchii, E5 Sanguisorba officinalis L, U1=Artemisia argyi Levl. et Vant., PE Camellia sinensis var. assamica, HJT-1933=Rhodiola rosea L, Z-12=Taxillus sutchuenensis (Lecomte) Danser, T-P=total glucosides of white paeony, and E1=Scutellaria baicalensis Georgi.

### Compositions

The present invention relates to compositions comprising extract from at least one herb selected from the group consisting of Caesalpinia sappatin L (A16), Salvia miltiorrhiza Bge (B8), Glycyrrhiza uralensis Fisch (G), Punica granatum L (PM), Rubia cordifolia L. (Y1830), Inula japonica, (capitulum) (P8), Antrodia camphorata (Y1646), Apocynum venetum L (F1), Eurycoma longifolia (TK), Paeonia veitchii (X7), Sanguisorba officinalis L (E5), Artemisia argyi Levl. et Vant. (U1), Camellia sinensis var. assamica (PE), Rhodiola rosea L (HJT-1933), Taxillus sutchuenensis (Lecomte) Danser (Z-12), total glucosides of white paeony (T-P), and Scutellaria baicalensis Georgi (E1).

The herbal extracts of the invention are useful within the methods of the invention, namely for treating or preventing the diseases and disorders that are related to the activity of TGF-β and/or IL-1β, and/or TNF-α, and/or IL6 are caused by bacterial, fungal or viral infection, or are inflammation-associated.

In certain embodiments, the herbal extracts of the invention are water extracts. The water extracts are prepared using a method comprising drying the herbs, grinding the dried herbs into an herb powder, adding the herb powder to an amount of water to form a mixture, heating the mixture to an elevated temperature for a period of time, allowing the mixture to cool down to room temperature, removing and removing any undissolved solids

In certain embodiments, the herbal powder is added to the water in a ratio of about 100 mg of herbs: 1 mL of water. In certain embodiments, the mixture is heated to a temperature of about 85ºC for about 30 mins. In certain embodiments, the undissolved solids are removed by centrifuging the mixture to form a pellet and then decanting and collecting the water extract, leaving behind the solid pellet.

### Combination Therapies

In certain embodiments, the extracts of the invention are useful in the methods of present invention when used concurrently with at least one additional agent that has anti-TGF-β and/or anti-IL-1β, and/or anti-TNF-α activity, and/or IL6 activity.

In certain embodiments, the additional agent is selected from the group consisting of Canakinumab, Pirfenidone, Nintedanib, Saracatinib, Etanercept, Infliximab, Adalimumab, Certolizumab, Tocilizumab, Sarilumab, Rilonacept, Anakinra and Bortezomib.

In certain embodiments, administration of the herbal extracts increases the efficacy of the anti-TGF-β and/or anti-IL-1β, and/or anti-TNF-α agent and/or anti-IL6 agent. In certain embodiments, the administration of the herbal extracts decreases the required effective dose or shortens the required dosage period of the anti-TGF-β and/or anti-IL-1β, and/or anti-TNF-α agent and/or anti-IL6 agent.

A synergistic effect can be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55).

### Methods of Treatment

In one aspect, the invention provides a method of treating or preventing a disease or disorder in a subject, the method comprises administering to the subject in need thereof a therapeutically effective amount of composition comprising at least one herbal extract or any active chemical present in the at least one herbal extract.

By the term “active chemical” as used herein is meant a chemical obtained from the at least one herbal extract where the chemical possesses a biological activity similar to that in the herbal extract, i.e., wherein the chemical inhibits the activity of TGF-β (and SMAD2/3) and/or IL-1β and/or TNF-α (and NF-kB), and/or IL6 (and STAT3).

In certain embodiments, the at least one herbal extract is from an herb selected from the group consisting of: Caesalpinia sappatin L. (A16), Salvia miltiorrhiza Bge (B8), Glycyrrhiza uralensis Fisch (G), Punica granatum L. (PM), Rubia cordifolia L. (Y1830), Inula japonica, (capitulum) (P8), Antrodia camphorata(Y1646), Apocynum venetum L. (F1), Eurycoma longifolia (TK), Paeonia veitchii (X7), Sanguisorba officinalis L (E5), Artemisia argyi Levl. et Vant. (U1), Camellia sinensis var. assamica (PE), Rhodiola rosea 1. (HJT-1933), Taxillus sutchuenensis (Lecomte) Danser (Z-12), total glucosides of white paeony (T-P) and Scutellaria baicalensis Georgi (E1).

In certain embodiments, the disease or disorder is related to the activity of TGF-β(and SMAD2/3) and/or IL-1β(and NF-kB), and/or TNF-α (and NF-kB), and/or IL6 (and STAT3). In certain embodiments, the disease or disorder is caused by bacterial, fungal, or viral infection. In certain embodiments, the disease or disorder is inflammation and/or fibrosis. In certain embodiments, the disease or disorder is inflammation and/or fibrosis or is associated with inflammation and/or fibrosis.

In certain embodiments, administering the composition inhibits the activity of TGF-β (and SMAD2/3) and/or IL-1β and/or TNF-α (and NF-kB), and/or IL6 (and STAT3).

In certain embodiments, the IC50 value of the composition ranges from about 20 μg/ml to about 760 μg/ml. In certain embodiments, the IC50 value of the composition is about 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, or about 760 μg/ml.

In certain embodiments, the disease or disorder related to the activity of TGF-β includes, for example, lung fibrosis, kidney fibrosis and/or liver fibrosis.

In certain embodiments, the disease or disorder related to the activity of IL-1β and/or TNF-α and/or IL6 includes, for example, lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and/or kidney cancer.

In certain embodiments, the disease or disorder caused by inflammation includes, for example, lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and/or kidney cancer.

In certain embodiments, the disease or disorder caused by bacterial or viral or fungal infection includes, for example, organ failure or disorder of blood cells.

In certain embodiments, the extracts of the invention co-administered concurrently with at least one additional agent that has anti-TGF-β and/or anti-IL-1β, and/or anti-TNF-α and/or IL6 activity. In other embodiments, the one or more herbal extracts are administered to the subject before administering the at least one additional agent that has anti-TGF-β and/or anti-IL-1β, and/or anti-TNF-α, and/or IL6 activity. In yet embodiments, the one or more herbal extracts are administered to the subject after administering at least one additional agent that has anti-TGF-β and/or anti-IL-1β, and/or anti-TNF-α and/or IL6 activity.

In certain embodiments, the additional agent is selected from the group consisting of canakinumab, Pirfenidone, nintedanib saracatinib, Etanercept, Infliximab, Adalimumab, Certolizumab, Tocilizumab, Sarilumab, Rilonacept, Anakinra, and Bortezomib.

In certain embodiments, the one or more herbal extracts of the invention are co-administered concurrently with at least one additional agent that has anti-bacterial, anti-fungal and/or anti-viral activity. In other embodiments, the one or more herbal extracts are administered to the subject before administering the at least one additional agent that has that has anti-bacterial, anti-fungal and/or anti-viral activity. In yet other embodiments, the one or more herbal extracts are administered to the subject after administering at least one additional agent that has that has anti-bacterial, anti-fungal and/or anti-viral activity.

In certain embodiments, the therapeutically effective amount of the additional agent that has anti-TGF-β and/or anti-IL-1β, and/or anti-TNF-α and/or IL6 activity is lowered when administered as part of the methods of the invention to a greater extent than if it was administered alone. In other embodiments, the therapeutically effective course of treatment with the additional agent that has anti-TGF-β and/or anti-IL-1β, and/or anti-TNF-α and/or IL6 activity is shorter when administered as part of the methods of the invention than if it was administered alone.

In certain embodiments, the composition is administered to the subject by at least one route selected from the group consisting of oral, nasal, inhalational, topical, buccal, rectal, pleural, peritoneal, intra-peritoneal, vaginal, intramuscular, subcutaneous, transdermal, epidural, intratracheal, optic, intraocular, intrathecal, and intravenous routes.

In certain embodiments, the composition is administered in a form selected from the group consisting of a pill, tablet, capsule, soup, tea, concentrate, dragees, liquids, drops, and gelcaps.

In certain embodiments, the composition is administered orally or nasally as liquid spray or aerosolized formulation.

In certain embodiments, the therapeutically effective amount of the herbal composition is about 0.5 g/day to about 10 g/day.

In certain embodiments, the subject is a mammal.

In certain embodiments, the subject is a human.

### Administration/Dosage/Formulations

The regimen of administration may affect what constitutes an effective amount. The therapeutic formulations may be administered to the subject either prior to or after the onset of disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 1,000 mg/kg of body weight/per day. The pharmaceutical compositions useful for practicing the invention may be administered to deliver a dose of from 1 ng/kg/day and 100 mg/kg/day. One of ordinary skill in the art would be able to study the relevant factors and make the determination regarding the effective amount of the therapeutic compound without undue experimentation.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the invention.

In certain embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier. In yet other embodiments, the compound of the invention is the only biologically active agent (i.e., capable of treating cancer) in the composition. In yet other embodiments, the compound of the invention is the only biologically active agent (i.e., capable of treating cancer) in therapeutically effective amounts in the composition.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds and/or compositions of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 400 mg to about 6,000 mg, about 500 mg to about 5,000 mg, about 750 mg to about 4,000 mg, about 1,000 mg to about 3,000 mg, about 1,000 mg to about 2,500 mg, about 20 mg to about 2,000 mg and any and all whole or partial increments therebetween. In certain embodiments, the dose of the compounds and/or compositions of the invention is about 800 mg.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents. Routes of administration of any of the compositions of the invention include oral nasal, rectal, intravaginal, parenteral, buccal, sublingual, or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-peritoneal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are soups, teas, concentrates, tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation”. For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of a disease or disorder contemplated in the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intra-peritoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1, 3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds useful within the methods of the invention may be administered in the form of microparticles, for example by injection, or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present invention depends on the age and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods:

### Standardized Preparation of Herbal Extracts

100 mg herbal powder was dissolved in 1 mL ultra-pure water (5 mL, resistance >15 mega (2) at 85° C. for 30 minutes. The aqueous fraction was then centrifuged. Supernatant was transferred to new 1.5 ml tube and stored at −20° C. in the dark until use.

### Luciferase Assay for NFkB Activity, SMAD2/3 Activity STAT3 Activity

HEK-293 and A549 reporter cells were stably transfected with PGL4 reporters carrying NFkB response element DNA: gggaatttcc×4, SEQ ID NO: 1, or carrying SMAD2/3 response element DNA: gagtatgtctagact×4), SEQ ID NO: 2, and STAT3 response element DNA: tgcattcccgtaa×6, SEQ ID NO: 3. Cells were seeded into haft-area 96-well microplate at 20000 cells/well in 40 μL of medium overnight at 37° ° C., and 5% CO₂ atmosphere in an incubator. Another dose of herbal water extract from 750 μg/mL to 83 μg/mL was added to the cells and placed in 37° C. with a 5% CO₂ atmosphere in an incubator with or without TNF-α (10 ng/mL) or IL1b (2.5 ng/mL) or TGFb (2.5 ng/ml) or IL5 (20 ng/mL). After removing the medium after 6 hours, 10 μL of lysis buffer (Tris-HCl 25 mM at pH 7.8, DTT 2 mM, CDTA 2 mM, glycerol 10%, Triton X-100 1%) was used to lyse the cells and 40 μL of luciferase reaction buffer (Tris-HCl 20 mM at pH 7.8, NaHCO₃ 1 mM, MgSO₄ 2.5 mM, DTT 10 mM, Coenyzme-A lithium 60 μM, potassium luciferin 225 μM, ATP 250 μM) was added for reading luminescence using a luminescence microplate reader. IC50 (concentration required to inhibit 50% of control) or EC50 (concentration required to achieved 50% of maximum activation) was determined based on the dose-response curve.

### Cytokine Analysis by Cytometric Bead Array

THP1 cells were cultured in RPMI1640 plus 5% FBS. THP1 cells at a density of 10⁶ cells/well on a 12-well plate were differentiated with TPA (10 ng/ml) for 48 h. After exchanging the differentiation medium, herbal water (250 μg/mL, 500 μg/mL, or 750 μg/mL) was added together with LPS 1 μg/mL to the cells with 2 mL of medium. Wells without LPS were used as negative control and wells containing only LPS were used as the positive control. After 24 h hours following the treatments, the medium of the wells was transferred to a 1.5 mL tube and centrifuged at 4000 rpm for 2 min. The supernatant was used for cytokine detection. Cytokine expression (IL-6, MIP-la, IL-5, IL-17A, IL-12p70, TNF-α, IL-1β, IL-10, MIG, IFN-γ, MCP-1, G-CSF) was performed using cytometric bead array flex set kit by flow cytometry (BD Canto II, New Jersey, USA) according to the manufacturer's instructions (BD biosciences, UK).

### Western Blot Protocol

Total cell lysis was prepared using 2×SDS sample buffer (62.5 mM Tris-HCl, 2% SDS, 10% glycerol, 50 mM DTT, and 0.05% bromophenol blue) and sonicated for 10 s to shear the DNA. Cell nuclei were isolated using Tris buffer saline with 0.4% NP40. Cell extracts were then subjected to electrophoresis through 10% SDS-polyacrylamide gels and transferred to 0.2 μm nitrocellulose membranes (Bio-Rad Laboratories, Hercules, CA) with a Miniprotein II transferring apparatus (Bio-Rad). The membranes were blocked and probed in TBS-T buffer (1×TBS buffer, 0.2% Tween 20) containing 5% non-fat milk. Anti-COX2, Anti-IL-1β, Anti-LOXL2, Anti-TGM2 were purchased from Cell Signaling Inc. Anti-GAPDH was used to detect GAPDH as the internal control to confirm equal protein loading. The membranes were then incubated with horseradish peroxidase-conjugated anti-mouse IgG and anti-rabbit IgG (1:5,000; Sigma). Enhanced chemiluminescence reagents (Perkin-Elmer Life Science Products, Boston, MA) were used to visualize the immunoreactive bands and the densities of protein bands were scanned using and analyzed using ImageJ software from the NIH.

### LPS Induced Inflammation Model in Mice

The selected herbs have in vivo activity against LPS induced inflammation. LPS (2.5 mg/kg, IP (intraperitoneal injection), once, Lipopolysaccharides from Pseudomonas aeruginosa 10) was injected into 10 week old female BDF1 mice. Water was injected for negative control. After LPS IP for 30 minutes, water (LPS control group) or herbal water extract (LPS+herb groups) (1 g/kg for TP, PM, F1, P8, E5 or 500 mg/kg for A16, U1, Y1830, P.O. BID) was fed orally to the mice (about 11 AM and 4 pm). After LPS IP for 24 hr, blood was taken for a complete blood count. Plasma was collected for pro-inflammatory cytokine/chemokine quantification using bead array assays. mRNA of different tissues (lung, kidney, liver, spleen) was extracted for qRT-PCR for quantifying the pro-inflammatory gene expression.

### Real Time Quantitative PCR (RT-qPCR) for Inflammation Associated Gene

Tissue, about 200 mg, was homogenized in a 2 mL tube with 1 mL Trizol and 200 μL chloroform. After 5 min of centrifugation at 10000 rpm, 0.5 mL supernatant was mixed with 0.5 mL 100% EtOH. RNA was isolated using the Roche High Pure RNA isolation kit. cDNA was then generated from RNA samples using Bio-rad iScript Advanced cDNA synthesis kit for RT-qPCR. qPCR was performed using specific for mice MCP1, IL-1β, TNF-α, IL6, CXCL1, CXCL9, CXCL10, iNOS and GAPDH primer (as shown in the table below) and iTaq™ Universal SYBR® Green Supermix in CFX PCR machine (Bio-rad). Relative mRNA expression was calculated based on the change of the threshold cycle relative to the internal control, GAPDH, using a standard curve generated by purified PCR products.

| Gene | Forward primer | Reverse primer |
|---|---|---|
| MCP1 | GGCTGGAGCATCCACGTGTT | TGGTGACAAAAACTACAGC |
| | (SEQ ID NO: 4) | (SEQ ID NO: 5) |
| IL-1β | AGGTCAAAGGTTTGGAAGCA | TGAAGCAGCTATGGCAACTG |
| | (SEQ ID NO: 6) | (SEQ ID NO: 7) |
| TNF-α | CGGTGCCTATGTCTCAGCCTCTT | AGGCCATTTGGGAACTTCTCATC |
| | (SEQ ID NO: 8) | (SEQ ID NO: 9) |
| IL6 | AGAAAAGAGTTGTGCAATGGCAATTC | AATAGGCAAATTTCCTGATTATAT |
| | (SEQ ID NO: 10) | (SEQ ID NO: 11) |
| CXCL1 | GCCTATCGCCAATGAGCTGC | CTTGAGTGTGGCTATGACTTC |
| | (SEQ ID NO: 12) | (SEQ ID NO: 13) |
| CXCL9 | GGAGTTCGAGGAACCCTAGTG | GGGATTTGTAGTGGATCGTGC |
| | (SEQ ID NO: 14) | (SEQ ID NO: 15) |
| CXCL10 | CAAAAGTAACTGCCGAAGCA | CTGAGCTAGGGAGGACAAGG |
| | (SEQ ID NO: 16) | (SEQ ID NO: 17) |
| iNOS | CAGAAGCAGAATGTGACC | GTAGTAGTAGAATGGAGATAGG |
| | (SEQ ID NO: 18) | (SEQ ID NO: 19) |
| GAPDH | GGGCTGCCATTTGCAGTGGCAA | TGTTAGTGGGGTCTCGCTCCTG |
| | (SEQ ID NO: 20) | (SEQ ID NO: 21) |

### Example 1: Identifying Herbs with Inhibitory Activity on IL-1β-NF-κB, TNF-α-NF-kB, TGF-β-SMAD2/3, IL-1β+TGF-β-SMAD2/3 TNF-α+TGF-β-SMAD2/3 Mediated Transcriptional Activity

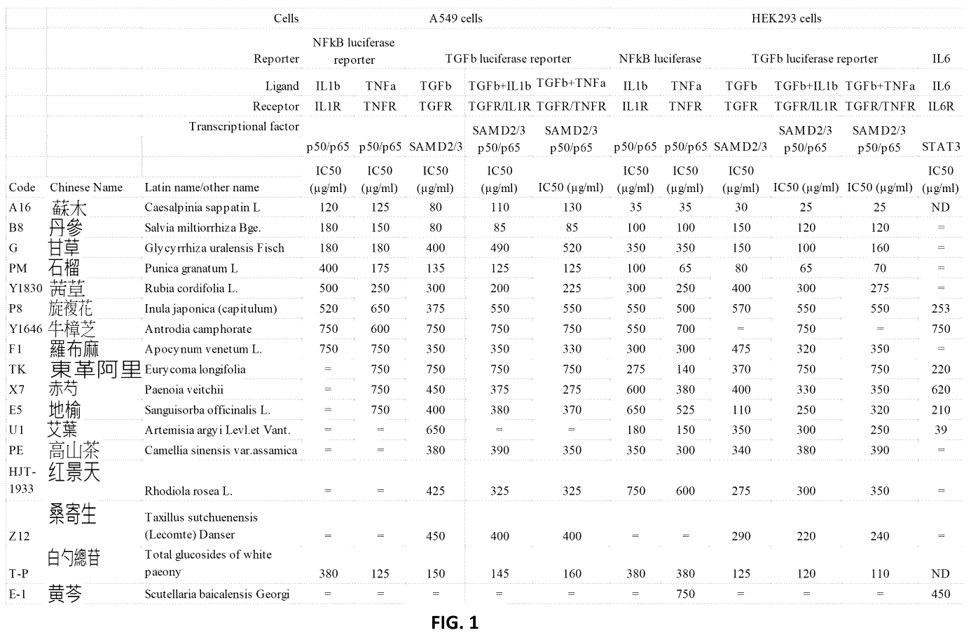

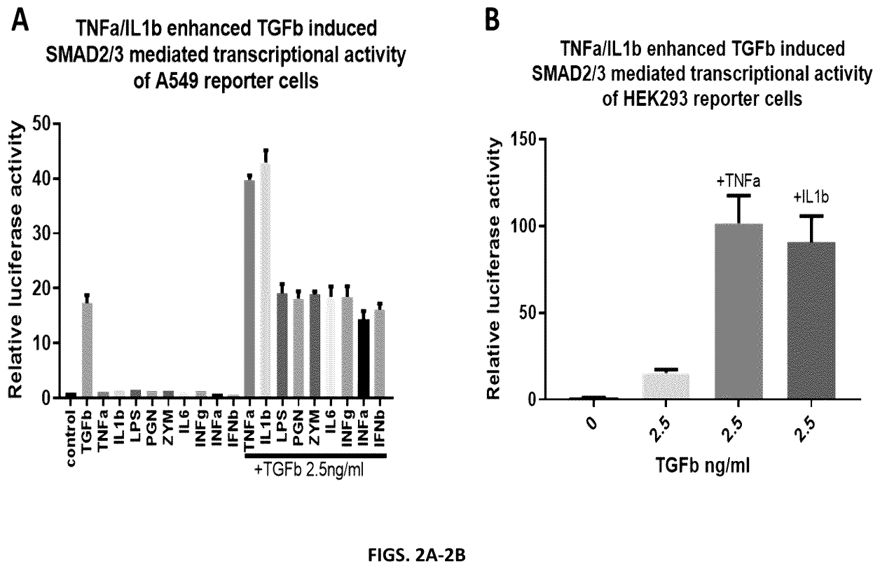

By STAR database analysis, herbs or their extracts that inhibited IL-1β-NF-κB, TNF-α-NF-kB, TGF-β-SMAD2/3, IL6-STAT3 mediated transcriptional activity were ranked (FIG. 1). Further, it was found that TNF-α or IL-1β had potential to enhance TGF-β induced SMAD2/3 mediated transcriptional activity (FIGS. 2A-2B). Therefore, the IC50 of the selected herb for inhibiting TGF-β plus IL-1β or TGF-β plus TNF-α mediated SMAD2/3 mediated transcriptional activity (FIG. 1) was also determined. As shown in the FIG. 1, A16, B8, G, PM, Y1830, P8, Y1646, F1, TK, X7, E5, U1, PE, HJT-1933, T-P, E1 showed activity against IL-1β or TNF-α induced NF-kB activity of HEK293 cells (primary embryonic human kidney cells). For A549 cells (adenocarcinoma human alveolar basal epithelial cells), A16, B8, G, PM, Y1830, P8, Y1646, F1, T-P inhibited IL-1β action while A16, B8, G, PM, Y1830, P8, Y1646, F1, TK, X7, E5, U1, T-P inhibited TNF-α action. In various embodiments, the aforementioned active herbs (used singly or in combination) can prevent or treat inflammation-associated diseases.

A16, B8, G, PM, Y1830, P8, Y1646, F1, TK, X7, E5, PE, HJT-1933, Z12, T-P inhibited TGF-β, TGF-β plus IL-1β or TGFB plus TNF-α induced SMAD2/3 mediated transcriptional response of A549 cells (FIG. 1). U1 only inhibit TGF-β alone action but not TGF-β plus IL-1β or TGF-β plus TNF-α action of A549 cells. A16, B8, G, PM, Y1830, P8, F1, TK, X7, E5, PE, HJT-1933, Z12, U1, T-P could inhibit TGF-β, TGF-β plus IL-1β or TGFB plus TNF-α induced SMAD2/3 mediated transcriptional response of HEK293 cells (FIG. 1). Y1646 only inhibit TGF-β plus IL-1β action but not TGF-β alone or TGFB plus TNF-α action of HEK293 cells. In various embodiments, the aforementioned active herbs (used singly or in combination) can prevent or treat diseases due to hyperactive TGF-β signaling.

P8, Y1646, TK, X7, E5, U1, E1 could inhibit IL6 induced STAT3 mediated transcriptional response of HEK293 cells (FIG. 1.) All these active herbs (single use or in combination) had potential to prevent or treat inflammation-associated diseases.

Combination of active herbs that could inhibit IL-1β, TNF-α, IL6 and TGFb action could better systemic effect on inflammation and fibrotic diseases.

### Example 2: Western Blot for COX2 and ICAM for Validating the Inhibition Effect of the Selected Herbs on IL-1β or TNF-α Action of A549 Cells

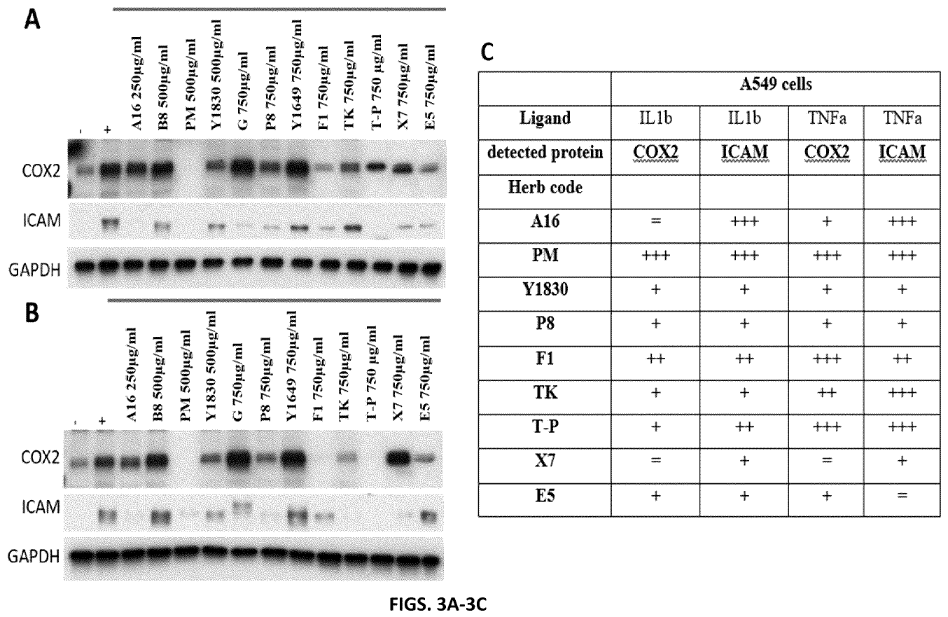

Western blotting analyses were performed to validate the inhibition of NF-kB target protein expression of A549 cells by the selected herbs. The results indicated A16, PM, Y1830, P8, F1, TK, T-P, X7 and E5 showed varying degrees of inhibition on IL-1β or TNF-α triggered COX2, ICAM protein expression of A549 cells (FIGS. 3A-3B). The selectivity and the degree of inhibition on COX2 and ICAM protein given by the active herbs are summarized in FIG. 3C.

### Example 3: Western Blot for LOXL2 and TGM2 for Validating the Inhibition Effect of the Selected Herbs on TGF-β Action of A549 Cells

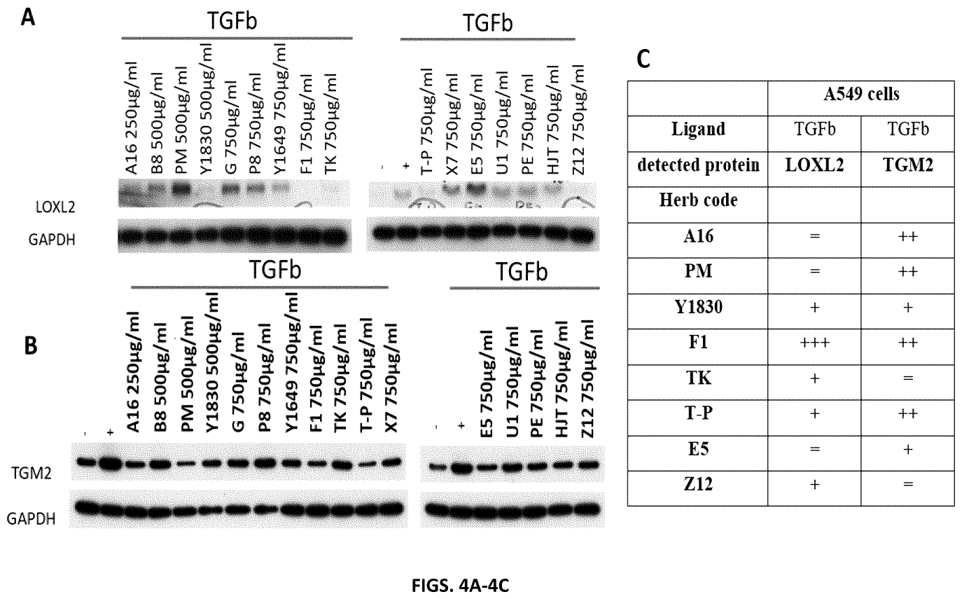

Western blotting analyses were performed to validate if the selected herbs can inhibit SMAD2/3 target protein expression of A549 cells. The results indicated A16, PM, Y1830, F1, TK, T-P, E5, Z12 showed varying degree of inhibition on TGF-β triggered LOXL2 (Lysyl Oxidase Homolog 2), TGM2 (transglutaminase 2) protein expression of A549 cells (FIGS. 4A-4B). The selectivity and the degree of inhibition of COX2 and ICAM protein by the active herbs is summarized in FIG. 4C.

### Example 4: Western Blot for COX2 and IL-1β for Validating the Inhibiting Effect of the Selected Herbs on LPS Action on THP1 Cells

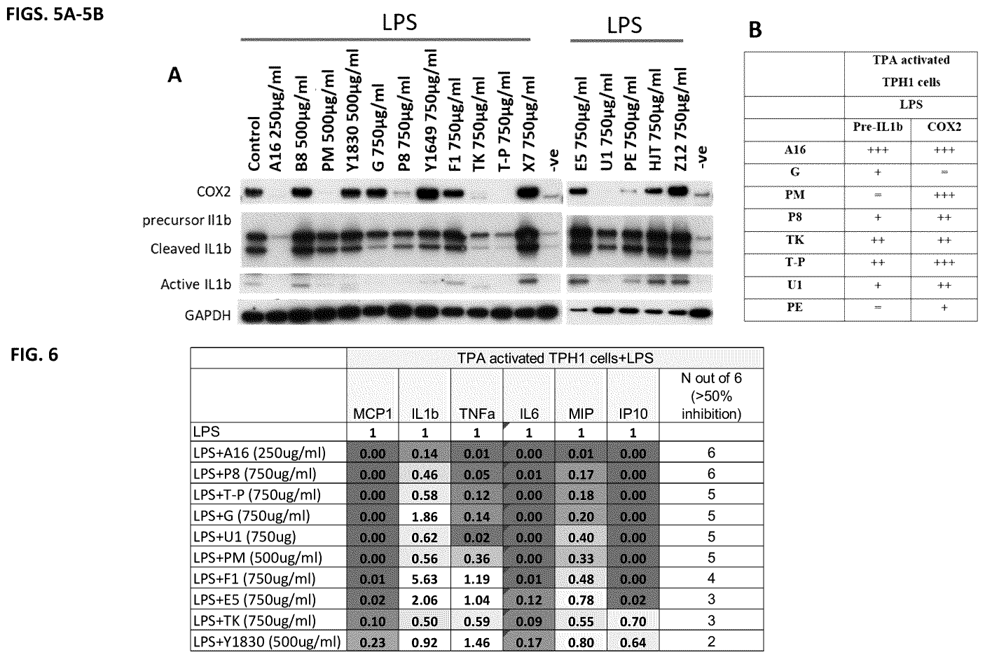

The immune cell model (TPA activated THP1 cells) was used to show if these selected herbs could also inhibit LPS triggered inflammation based on western blotting analysis. Results indicated A16, G, PM, P8, TK, T-P, U1, PE showed varying selectivity or varying of inhibition on LPS triggered COX2, IL-1β (precursor, cleaved, active IL-1β) of TPA activated TPH1 cells (FIG. 5A). The selectivity and the degree of inhibition on COX2 and IL-1β proteins by the active herbs is summarized in FIG. 5B.

### Example 5: Pro-Inflammatory Cytokine/Chemokine Detection for Validating the Inhibiting Effect of the Selected Herbs on the LPS Action on TPH1 Cells

The effect of the selected herbs on LPS induced cytokine/chemokine (IL1b, IL6, TNF-α, MCP1, MIP and IP10) secretion from TPA differentiated THP1 was further investigated. The results indicated that most of the selected herbs, at the concentration used, can inhibit (>50%) more than one cytokine/chemokines induced by LPS. There herbs were ranked according to the number of inhibited cytokine/chemokine as following A16, P8>T-P, G, U1, PM>F1>E5, TK, Y1830 (FIG. 6). In various embodiments, these herbs (A16, P8, T-P, G, U1, PM>F1>E5, TK, Y1830) can inhibit a “cytokine storm” induced by bacterial, viral, and/or fungi infection and could use for treating inflammation and fibrotic diseases.

### Example 6: Effect of the Selected Herbs on LPS Induced Pro-Inflammatory Gene Expression of Different Tissue in BDF1 Mice

The in vivo activity of selected herbs against LPS induced inflammation was assessed. LPS (2.5 mg/kg, IP, once, Lipopolysaccharides from Pseudomonas aeruginosa 10) were injected (intraperitoneal injection) into 10 week old female BDF1 mice. Water was injected as a negative control. After LPS IP for 30 minutes, water (LPS control group) or herbal water extract (LPS+herb groups) (1 g/kg for TP, PM, F1, P8, E5 or 500 mg/kg for A16, U1, Y1830, P.O., twice daily (b.i.d.)) was fed orally to the mice (about 11 am and 4 pm). After LPS IP for 24 hr, blood was taken for complete blood count. Plasma was collected for pro-inflammatory cytokine/chemokine quantification using bead array assays. mRNA of different tissues (lung, kidney, liver, spleen) was extracted for qRT-PCR for quantifying the pro-inflammatory gene expression.

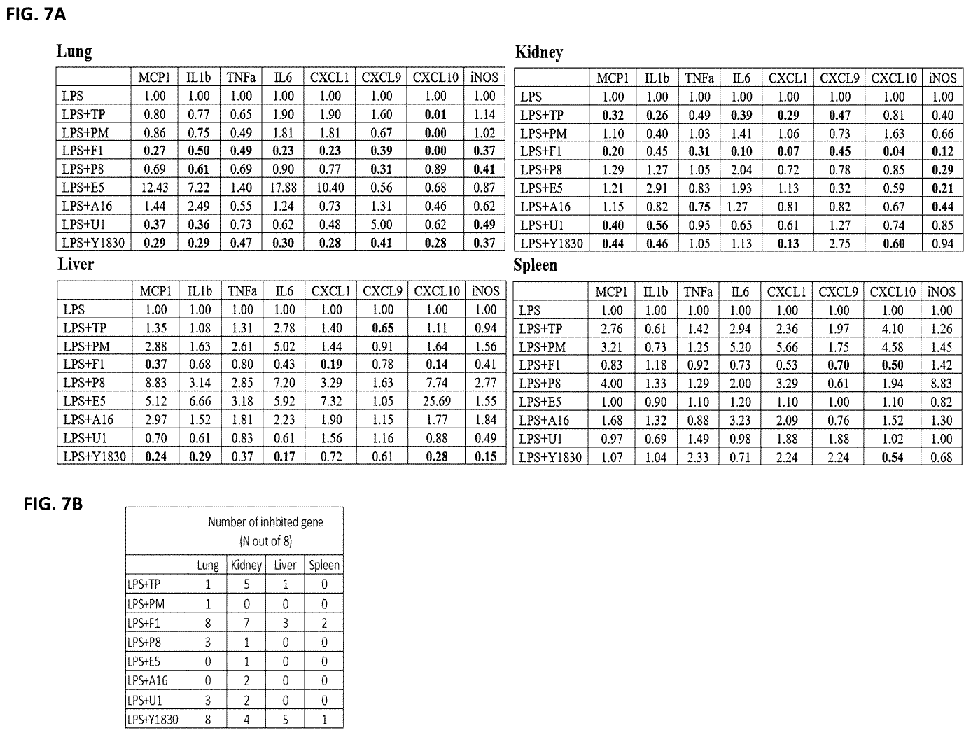

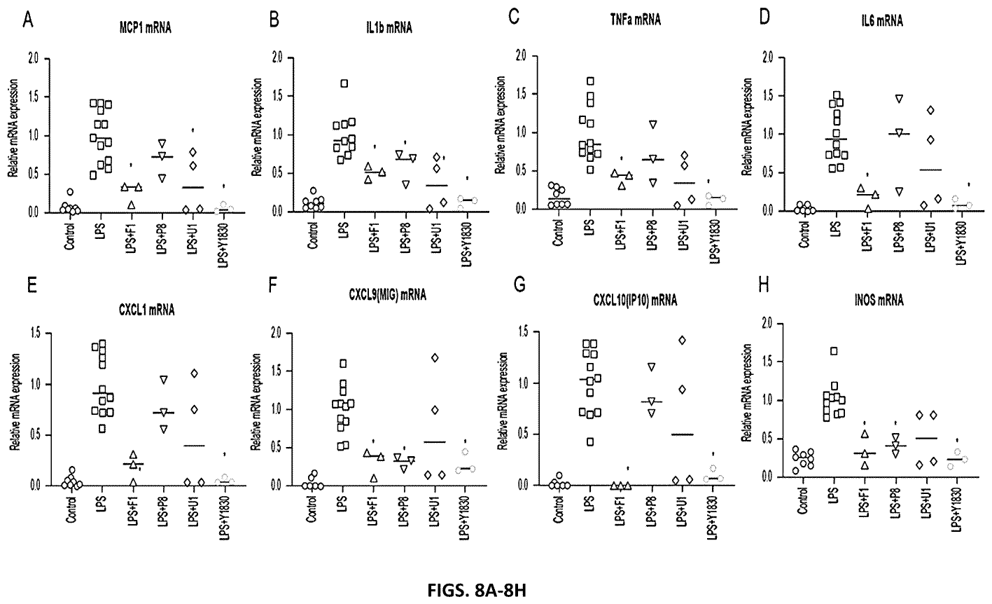

The effect of the selected herbs on the pro-inflammatory gene (MCP1, IL-1β, TNF-α, IL6, CXCL1, CXCL9 (MIG), CXCL10 (IP10), iNOS expression of different tissue following LPS treatment are shown in FIG. 7. The selected herbs were found to have tissue specific anti-inflammatory activity. F1 and Y1830 strongest impact on lung tissue. All tested genes (eight genes) of lung tissue were significantly inhibited by F1 and Y1830 (FIGS. 7A-7B and FIGS. 8A-8H). P8 and U1 could inhibit 3 out of 8 tested genes in lung tissue (FIGS. 7A-7B). F1 and Y1830 also had some impact on kidney as well as liver. T-P was quite selective on inhibitory pro-inflammatory gene induction in kidney tissue (FIGS. 7A-7B). For anti-lung inflammation, F1, Y1830, P8 and U1 can be used in therapies that treat or prevent inflammation in this organ. For anti-kidney inflammation, F1, Y1830 and T-P can be used in therapies that treat or prevent inflammation in this organ. In various embodiments, combinations of the selected herbs can be used to control or reduce whole-body inflammation.

### Example 7: Effect of the Selected Herbs on LPS Induced Pro-Inflammatory Cytokine/Chemokines Protein Expression of Plasma in BDF1 Mice

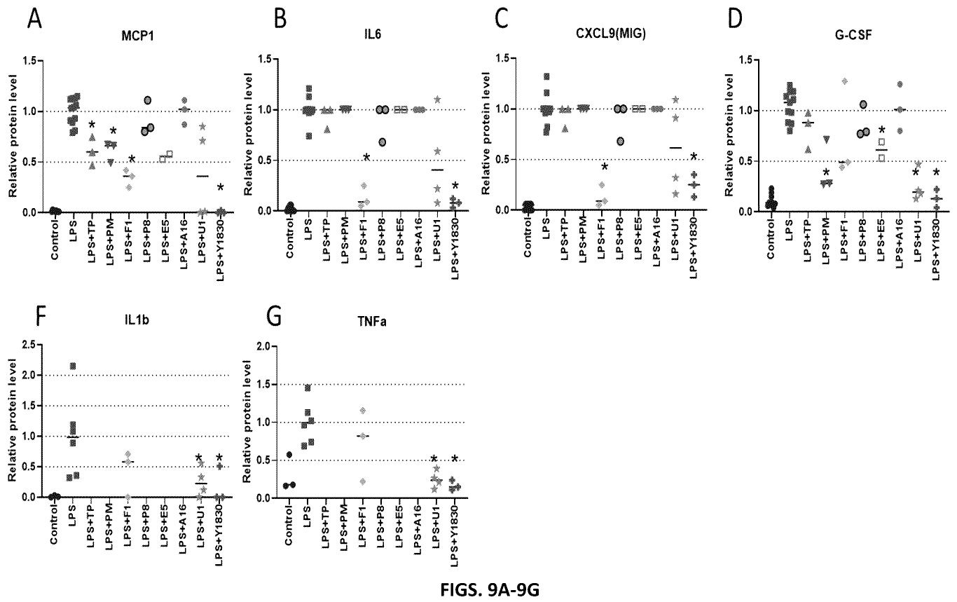

Bead array assay results showed F1 and Y1830 could inhibit MCP1, IL6, CXCL9, G-CSF, IL-1β, TNF-α in the plasma induced by LPS (FIGS. 9A-9G). PM could inhibit MCP1 and G-CSF (FIGS. 9A, 9D). TP could inhibit MCP1. These results demonstrated the active herbs could have systemic effect against LPS induced inflammation.

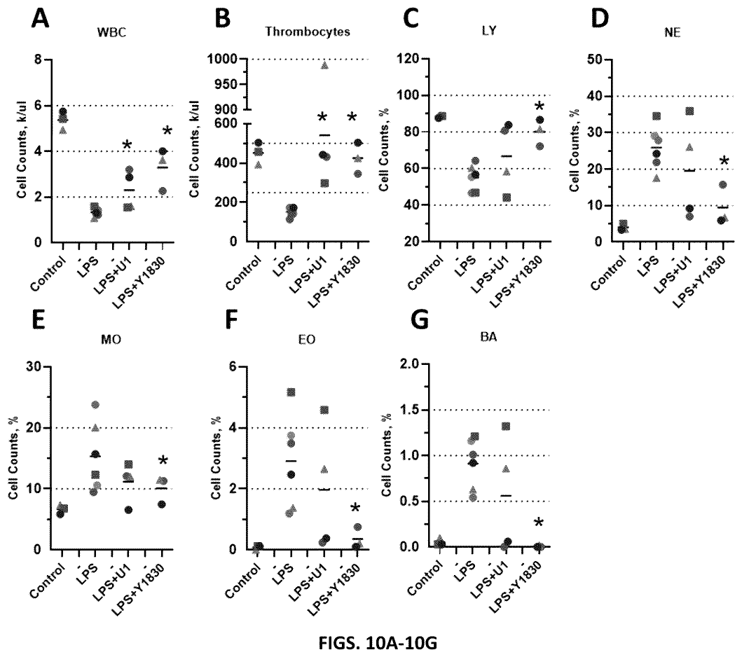

Results indicated that Y1830 could protect WBC, thrombocytes (or platelets), and lymphocytes drop induced by LPS (FIGS. 10A-10C). LPS induced inflammatory cells neutrophils, monocytes and eosinophils and basophile percentage of total blood. Y1830 could effectively inhibit all these inflammatory cells induced by LPS. U1 was found to inhibitory effect on WBC and lymphocytes drop induced by LPS.

### ENUMERATED EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. The following enumerated embodiments are provided, the numbering of which is not to be construed as designating levels of importance:

Embodiment 1 provides a method of treating or preventing a disease or disorder in a subject, the method comprising administering to the subject in need thereof a therapeutically effective amount of composition comprising at least one herbal extract or any active chemical present in the at least one herbal extract,
wherein the at least one herbal extract is from an herb selected from the group consisting of: Caesalpinia sappatin L. (A16), Salvia miltiorrhiza Bge (B8), Glycyrrhiza uralensis Fisch (G), Punica granatum L (PM), Rubia cordifolia L. (Y1830), Inula japonica, (capitulum) (P8), Antrodia camphorata (Y1646), Apocynum venetum L (F1), Eurycoma longifolia (TK), Paeonia veitchii (X7), Sanguisorba officinalis L (E5), Artemisia argyi Levl. et Vant. (U1), Camellia sinensis var. assamica (PE), Rhodiola rosea L (HJT-1933), Taxillus sutchuenensis (Lecomte) Danser (Z-12), total glucosides of white paeony (T-P), and Scutellaria baicalensis Georgi (E1); and
wherein the disease or disorder is
   (a) related to the activity of TGF-β (and SMAD2/3) and/or IL-1β and/or TNF-α (and NF-kB), and/or IL6 (and STAT3),
   (b) is caused by bacterial, fungal or viral infection, or
   (c) is the result of inflammation and/or fibrosis.

Embodiment 2 provides the method of embodiment 1, wherein the herbal extract or active chemical present therein inhibits the activity of TGF-β (and SMAD2/3) and/or IL-1β and/or TNF-α (and NF-kB), and/or IL6 (and STAT3).

Embodiment 3 provides the method of any one of embodiments 1-2, wherein the disease or disorder related to the activity of TGF-β (and SMAD2/3) is at least one from the group consisting of lung fibrosis, kidney fibrosis, and liver fibrosis.

Embodiment 4 provides the method of any one of embodiments 1-3, wherein the disease or disorder related to the activity of IL-1β and/or TNF-α (and NF-kB), and/or IL6 (and STAT3) is at least one from the group consisting of lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

Embodiment 5 provides the method of any one of embodiments 1-4, wherein disease or disorder resulting from an inflammation is at least one from the group consisting of lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

Embodiment 6 provides the method of any one of embodiments 1-5, wherein the disease or disorder caused by bacterial, viral, or fungal infection is at least one from the group consisting of organ failure or disorder of blood cells or sepsis shock.

Embodiment 7 provides the method of any one of embodiments 1-6, wherein the composition is administered orally or nasally.

Embodiment 8 provides the method of any one of embodiments 1-7, wherein the composition is administered in a form selected from the group consisting of a pill, tablet, capsule, soup, tea, concentrate, dragees, liquids, drops, and gelcaps, liquid sprays, or aerosolized formulation.

Embodiment 9 provides the method of any one of embodiments 1-8, wherein the IC50 value of the composition ranges from about 20 μg/ml to about 760 μg/ml.

Embodiment 10 provides the method of any one of embodiments 1-9, wherein the therapeutically effective amount of the herbal composition is about 0.5 g/day to about 10 g/day.

Embodiment 11 provides the method of any one of embodiments 1-10, wherein the composition is administered twice daily.

Embodiment 12 provides the method of any one of embodiments 1-11, wherein the composition further comprises at least one additional agent selected from the group consisting of an anti-TGF-β, anti-SMAD2/3, anti-IL-1β, and anti-TNF-α, anti-NF-kB, anti-IL6 and anti-STAT3 agents.

Embodiment 13 provides the method of any one of embodiments 1-12, wherein the at least one anti-TGF-β, anti-SMAD2/3, anti-IL-1β, and anti-TNF-α, anti-NF-kB, anti-IL6 and anti-STAT3 agents is selected from the group consisting of Canakinumab, Pirfenidone, Nintedanib, Saracatinib, Etanercept, Infliximab, Adalimumab, Certolizumab, Tocilizumab, Sarilumab, Rilonacept, Anakinra, and Bortezomib.

Embodiment 14 provides the method of any one of embodiments 1-13, wherein the subject is a mammal.

Embodiment 15 provides the method of any one of embodiments 1-14, wherein the subject is a human.

Embodiment 16 provides a method of treating or preventing at least one disease or disorder related to the activity of TGF-β (and SMAD2/3) in a subject, the method comprising administering to the subject in need thereof a therapeutically effective amount of composition comprising at least one herbal extract or any active chemical present in the at least one herbal extract,
wherein the at least one herbal extract is from an herb selected from the group consisting of: Caesalpinia sappatin L (A16), Salvia miltiorrhiza Bge (B8), Glycyrrhiza uralensis Fisch (G), Punica granatum L (PM), Rubia cordifolia L. (Y1830), Inula japonica, (capitulum) (P8), Antrodia camphorata (Y1646), Apocynum venetum L (F1), Eurycoma longifolia (TK), Paeonia veitchii (X7), Sanguisorba officinalis L (E5), Artemisia argyi Levl. et Vant. ((1), Camellia sinensis var. assamica (PE), Rhodiola rosea L (HJT-1933), Taxillus sutchuenensis (Lecomte) Danser (Z-12), total glucosides of white paeony (T-P), and Scutellaria baicalensis Georgi (E1); and
wherein the at least one disease or disorder is inflammation and/or fibrosis or is associated with inflammation and/or fibrosis.

Embodiment 17 provides the method of embodiment 16, wherein the herbal extract or active chemical present therein inhibits the activity of TGF-β (and SMAD2/3).

Embodiment 18 provides the method of any one of embodiments 16-17, wherein the disease is a disease of kidney, lung and/or liver.

Embodiment 19 provides the method of any one of embodiments 16-18, wherein the disease or disorder is at least one selected from the group consisting of lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

Embodiment 20 provides the method of any one of embodiments 16-19, wherein the composition further comprises at least one additional anti-TGF-β agent.

Embodiment 21 provides a method of treating or preventing at least one disease or disorder caused by bacterial, fungal and/or viral infection in a subject, the method comprising administering to the subject in need thereof a therapeutically effective amount of composition comprising at least one herbal extract or any active chemical present in the at least one herbal extract,
wherein the at least one herbal extract is from an herb selected from the group consisting of: Caesalpinia sappatin L (A16), Salvia miltiorrhiza Bge (B8), Glycyrrhiza uralensis Fisch (G), Punica granatum L (PM), Rubia cordifolia L. (Y1830), Inula japonica, (capitulum) (P8), Antrodia camphorata (Y1646), Apocynum venetum L (F1), Eurycoma longifolia (TK), Paeonia veitchii (X7), Sanguisorba officinalis L (E5), Artemisia argyi Levl. et Vant. ((1), Camellia sinensis var. assamica (PE), Rhodiola rosea L (HJT-1933), Taxillus sutchuenensis (Lecomte) Danser (Z-12), total glucosides of white paeony (T-P), and Scutellaria baicalensis Georgi (E1).

Embodiment 22 provides the method of embodiment 21, wherein the disease or disorder is inflammation and/or fibrosis or is associated with inflammation and/or fibrosis.

Embodiment 23 provides the method of any one of embodiments 21-22, wherein the disease or disorder is at least one selected from the group consisting of lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

Embodiment 24 provides the method of any one of embodiments 21-23, wherein the composition further comprises at least one additional anti-bacterial, anti-fungal, and/or anti-viral agent.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating or preventing a disease or disorder in a subject, the method comprising administering to the subject in need thereof a therapeutically effective amount of composition comprising at least one herbal extract or any active chemical present in the at least one herbal extract,
wherein the at least one herbal extract is from an herb selected from the group consisting of: Caesalpinia sappatin L (A16), Salvia miltiorrhiza Bge (B8), Glycyrrhiza uralensis Fisch (G), Punica granatum L (PM), Rubia cordifolia L. (Y1830), Inula japonica, (capitulum) (P8), Antrodia camphorata (Y1646), Apocynum venetum L (F1), Eurycoma longifolia (TK), Paeonia veitchii (X7), Sanguisorba officinalis L (E5), Artemisia argyi Levl. et Vant. (U1), Camellia sinensis var. assamica (PE), Rhodiola rosea L (HJT-1933), Taxillus sutchuenensis (Lecomte) Danser (Z-12), total glucosides of white paeony (T-P), and Scutellaria baicalensis Georgi (E1); and
wherein the disease or disorder is
(a) related to the activity of TGF-β (and SMAD2/3) and/or IL-1β and/or TNF-α (and NF-kB), and/or IL6 (and STAT3),
(b) is caused by bacterial, fungal or viral infection, or
(c) is the result of inflammation and/or fibrosis.

2. The method of claim 1, wherein the herbal extract or active chemical present therein inhibits the activity of TGF-β (and SMAD2/3) and/or IL-1β and/or TNF-α (and NF-kB), and/or IL6 (and STAT3).

3. The method of claim 1, wherein the disease or disorder related to the activity of TGF-β (and SMAD2/3) is at least one from the group consisting of lung fibrosis, kidney fibrosis, and liver fibrosis.

4. The method of claim 1, wherein the disease or disorder related to the activity of IL-1β and/or TNF-α (and NF-kB), and/or IL6 (and STAT3) is at least one from the group consisting of lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

5. The method of claim 1, wherein disease or disorder resulting from an inflammation is at least one from the group consisting of lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

6. The method of claim 1, wherein the disease or disorder caused by bacterial, viral, or fungal infection is at least one from the group consisting of organ failure or disorder of blood cells or sepsis shock.

7. The method of claim 1, wherein the composition is administered orally or nasally.

8. The method of claim 1, wherein the composition is administered in a form selected from the group consisting of a pill, tablet, capsule, soup, tea, concentrate, dragees, liquids, drops, and gelcaps, liquid sprays, or aerosolized formulation.

9. The method of claim 1, wherein the IC50 value of the composition ranges from about 20 μg/ml to about 760 μg/ml.

10. The method of claim 1, wherein the therapeutically effective amount of the herbal composition is about 0.5 g/day to about 10 g/day.

11. The method of claim 1, wherein the composition is administered twice daily.

12. The method of claim 1, wherein the composition further comprises at least one additional agent selected from the group consisting of an anti-TGF-β, anti-SMAD2/3, anti-IL-1β, and anti-TNF-α, anti-NF-kB, anti-IL6 and anti-STAT3 agents.

13. The method of claim 12, wherein the at least one anti-TGF-β, anti-SMAD2/3, anti-IL-1β, and anti-TNF-α, anti-NF-kB, anti-IL6 and anti-STAT3 agents is selected from the group consisting of Canakinumab, Pirfenidone, Nintedanib, Saracatinib, Etanercept, Infliximab, Adalimumab, Certolizumab, Tocilizumab, Sarilumab, Rilonacept, Anakinra, and Bortezomib.

14. The method of claim 1, wherein the subject is a mammal.

15. The method of claim 14, wherein the subject is a human.

16. A method of treating or preventing at least one disease or disorder related to the activity of TGF-β (and SMAD2/3) in a subject, the method comprising administering to the subject in need thereof a therapeutically effective amount of composition comprising at least one herbal extract or any active chemical present in the at least one herbal extract,
wherein the at least one herbal extract is from an herb selected from the group consisting of: Caesalpinia sappatin L (A16), Salvia miltiorrhiza Bge (B8), Glycyrrhiza uralensis Fisch (G), Punica granatum L (PM), Rubia cordifolia L. (Y1830), Inula japonica, (capitulum) (P8), Antrodia camphorata (Y1646), Apocymum venetum L (F1), Eurycoma longifolia (TK), Paeonia veitchii (X7), Sanguisorba officinalis L (E5), Artemisia argyi Levl. et Vant. (U1), Camellia sinensis var. assamica (PE), Rhodiola rosea L (HJT-1933), Taxillus sutchuenensis (Lecomte) Danser (Z-12), total glucosides of white paeony (T-P), and Scutellaria baicalensis Georgi (E1); and
wherein the at least one disease or disorder is inflammation and/or fibrosis or is associated with inflammation and/or fibrosis.

17. The method of claim 16, wherein the herbal extract or active chemical present therein inhibits the activity of TGF-β (and SMAD2/3).

18. The method of claim 16, wherein the disease is a disease of kidney, lung and/or liver.

19. The method of claim 16, wherein the disease or disorder is at least one selected from the group consisting of lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

20. The method of claim 16, wherein the composition further comprises at least one additional anti-TGF-β agent.

21. A method of treating or preventing at least one disease or disorder caused by bacterial, fungal and/or viral infection in a subject, the method comprising administering to the subject in need thereof a therapeutically effective amount of composition comprising at least one herbal extract or any active chemical present in the at least one herbal extract,
wherein the at least one herbal extract is from an herb selected from the group consisting of: Caesalpinia sappatin L (A16), Salvia miltiorrhiza Bge (B8), Glycyrrhiza uralensis Fisch (G), Punica granatum L (PM), Rubia cordifolia L. (Y1830), Inula japonica, (capitulum) (P8), Antrodia camphorata (Y1646), Apocynum venetum L (F1), Eurycoma longifolia (TK), Paeonia veitchii (X7), Sanguisorba officinalis L (E5), Artemisia argyi Levl. et Vant. (U1), Camellia sinensis var. assamica (PE), Rhodiola rosea L (HJT-1933), Taxillus sutchuenensis (Lecomte) Danser (Z-12), total glucosides of white paeony (T-P), and Scutellaria baicalensis Georgi (E1).

22. The method of claim 21, wherein the disease or disorder is inflammation and/or fibrosis or is associated with inflammation and/or fibrosis.

23. The method of claim 21, wherein the disease or disorder is at least one selected from the group consisting of lung pneumonia, hepatitis, atherosclerosis, Alzheimer's diseases, Parkinson's diseases, nephritis, meningitis, encephalitis, lung fibrosis, liver fibrosis, kidney fibrosis, lung cancer, liver cancer and kidney cancer.

24. The method of claim 21, wherein the composition further comprises at least one additional anti-bacterial, anti-fungal, and/or anti-viral agent.
